(19) United States
(12) Patent Application Publication
(10) Pub. No.: US 2022/0162281 A1
(43) Pub. Date: May 26, 2022

(54) RECOMBINANT IRISIN GENE OPTIMIZED FOR EXPRESSION IN PLANTS AND METHOD FOR PRODUCING RECOMBINANT IRISIN PROTEIN USING SAME

(71) Applicant: BIOAPPLICATIONS INC., Gyeongsangbuk-do (KR)

(72) Inventors: Yong Jik Lee, Gyeongsangbuk-do (KR); Joo Huh, Gwangju (KR); Kyung Hee Park, Gyeonggi-do (KR)

(21) Appl. No.: 17/601,128

(22) PCT Filed: Apr. 1, 2020
(86) PCT No.: PCT/KR2020/004480

§ 371 Date: Oct. 4, 2021

(30) Foreign Application Priority Data
Apr. 2, 2019 (KR) 10-2019-0038211

(51) Int. Cl. C07K 14/575; A61P 3/00; C12N 15/82; A23L 33/185

(52) U.S. Cl. CPC C07K 14/575; A61P 3/00; A61K 38/00; A23L 33/185; C12N 15/8257

(57) ABSTRACT

The present invention relates to a recombinant irisin gene optimized for expression in plants and comprising a nucleotide sequence represented by SEQ ID NO: 1, a method for producing an irisin protein, and a composition for preventing or treating metabolic diseases, comprising same.

### TECHNICAL FIELD

The present invention relates to a recombinant irisin gene whose expression is optimized in plants, which includes a nucleotide sequence represented by SEQ ID NO: 1, a method for producing an irisin protein, and for preventing or treating a metabolic disease, which includes the same.

This application claims priority to Korean Patent Application No. 10-2019-0038211, filed Apr. 2, 2019, the disclosure of which is incorporated herein by reference in its entirety.

### BACKGROUND ART

Obesity is a metabolic disease that results from an imbalance between caloric intake and expenditure, and thus refers to a condition in which adipose tissue increases abnormally due to the excessive calorie intake. The causes of obesity include various factors such as genetic effects, environmental effects by a Western-style diet, psychological effects arising from stress, and the like, but an exact mechanism of occurrence and progression of obesity has not been clearly identified. The incidence of obesity continuously rises with a rapid increase in fat intake even in Korea. The National Health and Nutrition Examination Survey (KNHANES) has found that the fat intake ratio in the Korean diet more than doubled from 9.6% in 1990 to 19.5% in 2007, and an increase in animal fat intake was mainly responsible for such an increase in fat intake. In 2010, the number of obese Korean adults reached approximately 12 million people, one out of every three people. In particular, 1.61 million people are severely obese with a body mass index of 30 or more, which has doubled rapidly in the past decade.

Obesity has not only its own problems but also causes various disorders and complications such as hypertension, hyperinsulinemia, hyperlipidemia, fatty liver, atherosclerosis, cardiovascular disorders, obstructive sleep apnea, certain types of cancer, arthritis, and the like as the patients' state of obesity lasts. Therefore, the obesity is very dangerous because it ultimately shortens lives. Also, because the socioeconomic costs incurred due to obesity and complications caused by obesity are enormous, there is much interest in the treatment of obesity around the world. However, there is still no effective therapeutic agent for treating metabolic diseases caused by obesity or the excessive accumulation of fat.

Uncoupling protein-1 (UCP1) is a gene responsible for the generation of heat, and serves to generate heat in brown adipose tissue. UCP1 present in the mitochondrial membrane is a substance that allows energy to be wasted as heat by using hydrogen ions to generate heat while preventing the hydrogen ions from being used to synthesize ATP. UCP1 is known to be expressed in brown adipocytes in animals and serves to maintain body temperature. The heat generation by UCP naturally promotes energy consumption.

It has been reported that adiponectin secreted from fat cells shows various effects including an anti-diabetic effect. That is, adiponectin is effective for preventing diabetes by increasing insulin sensitivity to lower blood sugar. This adiponectin is reported to be a protein whose expression increases as fat cells differentiate. Therefore, a substance that increases the expression of adiponectin during a fat cell differentiation process may show a useful effect in prevention and treatment of metabolic diseases including obesity, diabetes, and the like.

Irisin, which is a glycosylated protein hormone composed of 112 amino acids, is formed by proteolytic cleavage of FNDC5. Production of FNDC5 is promoted by the exercise in muscles. In this case, the FNDC5 may be converted into PGC1a using a transcription coactivator PGC1a. Irisin is secreted from muscles, circulates to the adipose tissue, and regulates energy metabolism. It is known that irisin promotes a process of transforming white adipose tissue into brown adipose tissue. Irisin is synthesized in muscle tissue and is present in Purkinje cells of the cerebellum and the intercellular nerve endings.

Meanwhile, proteins for preventing the above disorders have been produced and developed, but are mainly produced using animal cells rather than bacterial cells due to the problems such as protein folding, glycosylation, and the like. However, a method for producing a protein using the animal cells has drawbacks in that it is not easy to prepare proteins, and vaccines are very expensive because it takes a lot of money to expand facilities for mass production of the proteins. Also, the proteins prepared using the animal cells have drawbacks in that it is not easy to store the proteins and the proteins are highly likely to be contaminated with viruses with which animals may be infected. However, plants are expected to be likely to stably produce inexpensive proteins because, unlike the animal cells, the plants are not very likely to be contaminated with the viruses with which animals may be infected, may be mass-produced at any time when the cultivation area is secured, and the proteins may be stored in a plant for a long period of time.

### DISCLOSURE

### Technical Problem

The present invention is designed to solve the problems of the related art, and thus it is an object of the present disclosure to provide a recombinant irisin gene capable of being effectively produced using a plant and showing a high effect on physiological activity, a method for preparing a recombinant irisin protein using the same, and a composition for preventing or treating a metabolic disease using the protein.

However, the technical objects of the present invention are not limited to the technical objects as described above, and other objects of the present invention which are not stated herein will become more apparent to those of ordinary skill in the art by describing exemplary embodiments thereof in detail.

### Technical Solution

The present invention provides a recombinant irisin gene whose expression is optimized in plants, which includes a nucleotide sequence represented by SEQ ID NO: 1. The functional equivalents of the recombinant irisin gene consisting of a nucleotide sequence represented by SEQ ID NO: 1 fall within the scope of the present invention. The functional equivalents refer to genes that have a sequence homology of at least 60% or more, preferably 70% or more, more preferably 80% or more, and most preferably 90% or more with respect to the nucleotide sequence as a result of addition, substitution, or deletion of bases, and thus have substantially the same activity as the gene including the nucleotide sequence represented by SEQ ID NO: 1. In this case, a gene sequence of the recombinant irisin protein is not limited as long as it can be stably produced using a plant.

Also, the present invention provides a recombinant vector including the recombinant irisin gene.

According to one exemplary embodiment of the present invention, the vector may have a promoter gene and a nucleotide sequence represented by SEQ ID NO: 1 operably connected thereto in this sequential order.

According to another exemplary embodiment of the present invention, the recombinant vector may be constructed to express a plant-derived irisin protein.

According to still another exemplary embodiment of the present invention, the promoter includes a cauliflower mosaic virus-derived 35S promoter, a cauliflower mosaic virus-derived 19S RNA promoter, a plant-derived actin protein promoter, a ubiquitin protein promoter, a cytomegalovirus (CMV) promoter, a simian virus 40 (SV40) promoter, a respiratory syncytial virus (RSV) promoter, an elongation factor-1 alpha (EF-1α) promoter, a pEMU promoter, a MAS promoter, a histone promoter, a Clp promoter, and the like, but the present invention is not limited thereto.

According to yet another exemplary embodiment of the present invention, the recombinant expression vector may further include a polynucleotide encoding a chaperone binding protein (BiP), a gene encoding a peptide composed of 6 histidines, and the like.

According to yet another exemplary embodiment of the present invention, the vector may include the cleavage map shown in FIG. 1.

According to yet another exemplary embodiment of the present invention, the vector may further include a gene encoding one or more selected from the group consisting of a chaperone binding protein (BiP) gene and a tagging gene.

Also, the present invention provides a transformant transformed with the vector.

According to one exemplary embodiment of the present invention, the transformant may preferably include microorganisms such as Escherichia coli, Bacillus sp., Salmonella sp., yeasts, and the like, insect cells, animal cells including cells of humans, mice, rats, dogs, monkeys, pigs, horses, cattle, and the like, Agrobacterium tumefaciens, plants, and the like. More preferably, the transformant may include food crops including rice, wheat, barley, corn, bean, potato, wheat, red bean, oats, and sorghum; vegetable crops including Arabidopsis thaliana, Korean cabbage, radish, red pepper, strawberry, tomato, watermelon, cucumber, cabbage, oriental melon, pumpkin, green onion, onion, and carrot; special crops including ginseng, tobacco, cotton, sesame, sugar cane, sugar beet, perilla, peanut, and rape; fruit trees including apple trees, pear trees, jujube trees, peach trees, grape vines, citrus trees, persimmon trees, plum trees, apricot trees, and banana trees; and flowering plants including rose, carnation, chrysanthemum, lily, and tulip. However, living organisms are not limited as long as they can be transformed with the vector of the present invention.

According to one exemplary embodiment of the present invention, the transformant may be a plant or a plant cell, but the present invention is not limited thereto.

In addition, the present invention provides a method for producing a recombinant irisin protein, which includes: (a) culturing the transformant; and (b) separating and purifying a recombinant irisin protein from the transformant or the culture broth. Preferably, the transformant may be a cell itself, a plant, or a culture product including the cell, and the culture broth may be preferably a culture broth from which cells are removed after the cells are cultured. However, the culture broth is not limited as long as it includes the recombinant protein of the present invention.

According to one exemplary embodiment of the present invention, the purification of step (b) may be performed using a water-soluble fraction, but the present invention is not limited thereto.

Furthermore, the present invention provides a pharmaceutical composition for preventing or treating a metabolic disease, which includes the plant-derived recombinant irisin protein produced by the method as an active ingredient.

Also, the present invention provides a food composition for preventing or ameliorating a metabolic disease, which includes the plant-derived recombinant irisin protein produced by the method as an active ingredient.

In addition, the present invention provides a method for preventing or treating a metabolic disease, which includes: administering the plant-derived recombinant irisin protein produced by the method into a subject.

Additionally, the present invention provides a use of the plant-derived recombinant irisin protein produced by the method for preventing or treating a metabolic disease.

Furthermore, the present invention provides a use of the plant-derived recombinant irisin protein produced by the method for preparing a medicament for treating a metabolic disease.

According to one exemplary embodiment of the present invention, the metabolic disease may preferably include obesity, diabetes, hypertension, hyperlipidemia, hypertriglyceridemia, hypercholesterolemia, fatty liver, atherosclerosis, or the like, but the present invention is not limited thereto.

According to another exemplary embodiment of the present invention, the recombinant irisin protein may include one or more selected from the group consisting of a glycosylated recombinant irisin protein and a non-glycosylated recombinant irisin protein, but the present invention is not limited thereto.

According to still another exemplary embodiment of the present invention, the recombinant irisin protein may include an amino acid sequence set forth in SEQ ID NO: 4, but the present invention is not limited thereto.

Also, the present invention provides a composition for inducing differentiation of brown adipocytes, which includes the plant-derived recombinant irisin protein produced by the method.

Further, the present invention provides a method for inducing the differentiation of white adipocytes into brown adipocytes, which includes: treating white adipocytes with the plant-derived recombinant irisin protein produced by the method.

### Advantageous Effects

The recombinant irisin protein of the present invention can be effectively expressed in a plant, and can be easily separated and purified because the recombinant irisin protein has high water solubility. Also, the recombinant irisin protein of the present invention is expected to be effectively used to treat metabolic diseases because the recombinant irisin protein has an effect of inducing an increase in expression of UCP1 and adiponectin.

### DESCRIPTION OF DRAWINGS

FIG. 1 is a diagram showing an arrangement of genes for expressing irisin in a plant according to one embodiment of the present invention.

FIG. 2 is a diagram showing the results of confirming the expression of irisin in a plant by Western blotting according to one embodiment of the present invention.

FIG. 3 is a diagram showing the results of separating and purifying a plant-derived recombinant irisin according to one embodiment of the present invention.

FIG. 4 is a diagram showing the results of confirming whether the recombinant irisin is glycosylated by treatment with endoglycosidase H according to one embodiment of the present invention.

FIG. 5 shows the results of determining a change in amount of a UCP-1 protein in 3T3-L1 fat cells by means of Western blotting after the cells are stimulated with the recombinant irisin protein according to one embodiment of the present invention.

FIG. 6 shows the results of determining a change in amount of an adiponectin protein in 3T3-L1 fat cells by means of Western blotting after the cells are stimulated with the recombinant irisin protein according to one embodiment of the present invention. (*p<0.05 vs control).

### BEST MODE

In the present invention, it was confirmed that, when an irisin gene composed of SEQ ID NO: 1 is used, an irisin protein having high physiological activity even in a plant may be efficiently produced and separated, and may also maintain the same physiological activity as existing irisin proteins. Therefore, the irisin protein of the present invention is expected to provide an inexpensive and stable irisin protein because the irisin protein is stably and effectively mass-produced.

In this specification, the term “recombinant irisin gene” may refer to an irisin gene that includes a nucleotide sequence represented by SEQ ID NO: 1, preferably an irisin gene that consists of a nucleotide sequence represented by SEQ ID NO: 1. Also, the gene variants fall within the scope of the present invention. Specifically, the gene may include a nucleotide sequence having a sequence homology of 70% or more, more preferably 80% or more, and most preferably 90% or more with respect to the nucleotide sequence represented by SEQ ID NO: 1. For example, the gene may include a polynucleotide having a sequence homology of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%.

In this specification, the term “expression vector” refers to a vector that may express a peptide or a protein that is encoded by heterogeneous nucleic acids inserted into the vector. Preferably, the expression vector refers to a vector constructed to express a recombinant irisin protein. The term “vector” refers to any medium for introducing and/or delivering bases into a host cell in vitro, ex vivo, or in vivo, and may be a replicon that may bind to another DNA fragment to induce replication of the bound fragment. Here, the term “replicon” refers to any genetic unit (for example, a plasmid, a phage, a cosmid, a chromosome, a virus, and the like) that serves as an autonomous unit of DNA replication in vivo, that is, is replicable by its own regulation. The recombinant expression vector of the present invention may preferably include a promoter that is a transcription initiation factor to which an RNA polymerase binds, any operator sequence for regulating transcription, a sequence encoding a suitable mRNA ribosome binding site and a sequence controlling the termination of transcription and translation, a terminator, and the like. More preferably, the recombinant expression vector of the present invention may include a 5′ UTR region gene of M17 for increasing a synthesis amount of a protein, a BiP gene for translocating a target protein into the endoplasmic reticulum, a tagging gene for easily isolating a recombinant protein, and the like, and, more preferably, may further include a selective marker gene such as an antibiotic-resistant for selecting a transformant, and the like.

The “BiP gene” is a portion of a BiP sequence that is used to translocate an expressed recombinant protein into the endoplasmic reticulum, and is preferably a gene including a nucleotide sequence represented by SEQ ID NO: 2_ and most preferably a gene composed of SEQ ID NO: 2. In this case, the BiP gene may include a nucleotide sequence having a sequence homology of 80% or more, more preferably 90% or more, and more preferably 95% or more with respect to the nucleotide sequence represented by SEQ ID NO: 2. The term “percent sequence homology” with respect to a polynucleotide is determined by comparing a comparison region with an optimally aligned sequence. In the comparison region, a portion of a polynucleotide sequence may further include additions or deletions (i.e., gaps), compared to a reference sequence (which does not include additions or deletions) for optimal alignment of the sequence. The “BIP gene” is used to translocate the expressed recombinant protein into the endoplasmic reticulum, and it cleaved after it is expressed in plant cells.

The term “tagging gene” may preferably use a peptide fragment composed of 1 to 10 histidines, that is, a His-tag. Most preferably, the tagging gene may be a gene to which a peptide fragment composed of 6 histidines is attached, and is represented by a nucleotide sequence represented by SEQ ID NO: 3. As such, the histidine residues are most widely used as a tag required for purification after a recombinant protein is expressed, and should have high specificity and have as little influence as possible on a structure of a desired protein. Preferably, the tagging gene may be composed of a peptide having 1 to 10 continuous histidines. In this case, because the tagging gene has a small size and has little influence on the original protein structure, the tagging gene is convenient in that it is not separately cleaved after the recombinant protein is produced. The tag may be attached to the N-terminus (front), the C-terminus (rear), or both of a target protein, depending on the type of vector, and the front or rear of the target protein may be chosen depending on what has an influence on the structure of the protein.

As one example, the selective marker gene may include a herbicide-resistant gene such as glyphosate or phosphinothricin; an antibiotic-resistant gene such as kanamycin, G418, bleomycin, hygromycin, chloramphenicol, or the like; an aadA gene; and the like. As one example, the promoter may include a pEMU promoter, a MAS promoter, a histone promoter, a Clp promoter, a cauliflower mosaic virus-derived 35S promoter, a cauliflower mosaic virus-derived 19S RNA promoter, a plant-derived actin protein promoter, a ubiquitin protein promoter, a cytomegalovirus (CMV) promoter, a simian virus 40 (SV40) promoter, a respiratory syncytial virus (RSV) promoter, an elongation factor-1 alpha (EF-1α) promoter, and the like. As one example, the terminator includes a nopaline synthase (NOS) terminator, a rice amylase RAmy1 A terminator, a phaseolin terminator, an octopine gene terminator of Agrobacterium tumefaciens, an E. coli rrnB1/B2 terminator, and the like, but the examples are merely for the purpose of illustration and the present invention is not limited thereto.

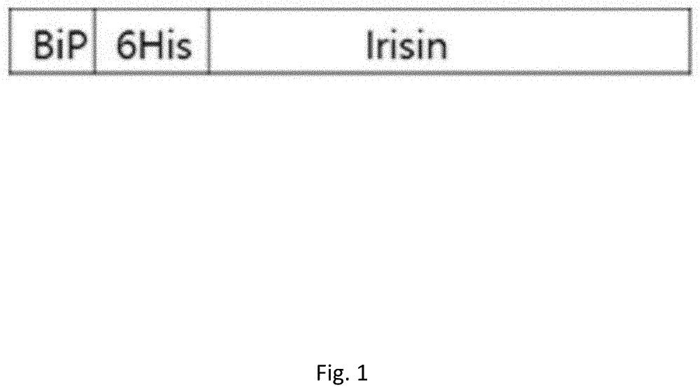

In this specification, the “vector” may include the structure map shown in FIG. 1, but the present invention is not limited thereto.

In this specification, the “vector” is preferably a gene including an amino acid sequence set forth in SEQ ID NO: 8, and most preferably may be composed of an amino acid sequence set forth in SEQ ID NO: 8, or include an amino acid sequence having a sequence homology of 80% or more, more preferably 90% or more, and most preferably 95% or more with respect to the sequence of SEQ ID NO: 8.

Also, the amino acid sequence of the vector may be encoded by a gene sequence set forth in SEQ ID NO: 7, but the present invention is not limited thereto. Specifically, the gene may include a nucleotide sequence having a sequence homology of 90% or more, more preferably 95% or more, and most preferably 98% or more with respect to the nucleotide sequence represented by SEQ ID NO: 7. The term “percent of sequence homology” with respect to a polynucleotide is determined by comparing a comparison region with an optimally aligned sequence. In the comparison region, a portion of a polynucleotide sequence may further include additions or deletions (i.e., gaps), compared to a reference sequence (which does not include additions or deletions) for optimal alignment of the sequence.

In this specification, the term “transformation” generally refers to a process in which genetic traits are changed by injection of DNA, and the term “transformant (i.e., a transgenic organism)” refers to a living organism prepared by injecting an exogenous gene using a molecular genetic method. Preferably, the transformant is a living organism transformed with the recombinant expression vector of the present invention, and the living organism is not limited as long as it is alive such as microorganisms, eukaryotic cells, insects, animals, plants, and the like. Preferably, the living organism includes E. coli, Salmonella sp., Bacillus sp., a yeast, an animal cell, a mouse, a rat, a dog, a monkey, a pig, horse, cattle, Agrobacterium tumefaciens, a plant, and the like, but the present invention is not limited thereto.

In this specification, plants may be used as the “plant” without any limitation as long they can mass-produce a protein. More specifically, the plant may be selected from the group consisting of tobacco, Arabidopsis thaliana, corn, rice, soybean, canola, alfalfa, sunflower, sorghum, wheat, cotton, peanut, tomato, potato, lettuce, and red pepper. Preferably, the plant may be tobacco. In the present invention, the tobacco is a plant belonging to the Nicotiana genus, and the type of tobacco is not particularly limited as long as it can overexpress a protein. In this case, the present invention may be put into practice by selecting a suitable variety depending on a transformation method, and a purpose of mass-producing a protein. For example, a variety such as Nicotiana benthamiana L., Nicotiana tabacum cv. xanthi, or the like may be used.

In this specification, the transformant may be a plant or a plant cell, but the present invention is not limited thereto.

The transformant may be prepared using methods such as transformation, transfection, an Agrobacterium-mediated transformation method, a particle gun bombardment method, a sonication method, an electroporation method, and a polyethylene glycol (PEG)-mediated transformation method, and the like. In this case, methods are not limited as long as they may be used to inject the vector of the present invention.

In this specification, the term “solubility” refers to the extent to which a target protein or peptide may be dissolved in a suitable solvent to be administered to the human body. Specifically, solubility may refer to the degree of saturation of a solute with respect to a given solvent at a certain temperature. Solubility may be measured by determining a saturation concentration of the solute. For example, an excessive amount of a solute is added to a solvent, stirred, and filtered. Then, a concentration of the solute may be measured using UV spectroscopy, HPLC, or the like, but the present invention is not limited thereto. Higher solubility is more favorable for separating and purifying a recombinant protein, and also advantageous for maintaining the physiological or pharmacological activity of the recombinant protein because it suppresses aggregation of the recombinant protein.

The “recombinant irisin protein” of the present invention includes an amino acid sequence set forth in SEQ ID NO: 4, and preferably is composed of an amino acid sequence set forth in SEQ ID NO: 4. According to one embodiment of the present invention, the recombinant irisin protein produced in a plant transformed with the vector including the recombinant irisin gene of the present invention may preferably include one or more selected from the group consisting of a glycosylated recombinant irisin protein and a non-glycosylated recombinant irisin protein, and more preferably, may include both of a glycosylated recombinant irisin protein and a non-glycosylated recombinant irisin protein, but the present invention is not limited thereto.

The term “glycosylation” used in the present invention is a process after protein translation in cells (eukaryotes), and is divided into N-glycosylation and O-glycosylation, which differ depending on the functional group attached to a protein. Here, a process of attaching a sugar (such as lactose and the like) to a protein produced in the cells is generally referred to “glycosylation.” When a sugar chain is linked to a protein via a glycosylation process, the protein undergoes a “folding” process to form a three-dimensional structure, which imparts stability to the protein so that a shape of the protein can be maintained without any unfolding for a long time. Also, the sugar chain attached to the protein is transferred to a cell membrane and becomes a cell membrane protein, and may also have the same effect as antigens. As described above, a glycosylated protein is referred to as a glycoprotein, and a representative example of the glycoprotein is an antibody that plays an important role in an immune response, and the like. A program (http://www.cbs.dtu.dk/services/NetNGlyc/) for predicting whether a protein is glycosylated is operated using an amino acid sequence of the vector of SEQ ID NO: 8 used in the present invention. As a result, it is predicted that the N-glycosylation occurs at asparagine (N) 16 or 61.

Therefore, the recombinant irisin protein used in the present invention may include one or more selected from the group consisting of a glycosylated recombinant irisin protein and a non-glycosylated recombinant irisin protein. In this case of the glycosylated recombinant irisin protein, the N-glycosylation occurs at asparagine (N) 8 and/or 53 of the recombinant irisin protein set forth in SEQ ID NO: 4, but the present invention is not limited thereto. Therefore, the recombinant irisin protein may be modified within a range having an equivalent effect to the present invention.

According to another aspect of the present invention, the present invention provides a pharmaceutical composition or preventing or treating a metabolic disease and a food composition for ameliorating a metabolic disease, both of which include the recombinant irisin protein.

According to still another aspect of the present invention, the present invention provides a composition for inducing differentiation of brown adipocytes, which includes the recombinant irisin protein.

According to yet another aspect of the present invention, the present invention provides a method for preventing or treating a metabolic disease, which includes: administering the plant-derived recombinant irisin protein produced by the method into a subject.

According to yet another aspect of the present invention, the present invention provides a use of the plant-derived recombinant irisin protein produced by the method for preventing or treating a metabolic disease.

Also, the present invention provides a use of the plant-derived recombinant irisin protein produced by the method for preparing a medicament for treating a metabolic disease.

According to one exemplary embodiment of the present invention, the metabolic disease may preferably include obesity, diabetes, hypertension, hyperlipidemia, hypertriglyceridemia, hypercholesterolemia, fatty liver, atherosclerosis, or the like, but the present invention is not limited thereto.

In addition, the present invention provides a composition for inducing differentiation of brown adipocytes, which includes the plant-derived recombinant irisin protein produced by the method.

Further, the present invention provides a method for inducing the differentiation of white adipocytes into brown adipocytes, which includes: treating the white adipocytes with the plant-derived recombinant irisin protein produced by the method.

In this specification, the term “metabolic disease” refers to a disorder that is caused by excessive synthesis or accumulation of fats as energy metabolism abnormally occurs in the body due to various causes such as excessive energy intake, hormone imbalance, and the like. Specifically, the metabolic disease may be obesity, diabetes, hypertension, hyperlipidemia, hypertriglyceridemia, hypercholesterolemia, fatty liver, or atherosclerosis.

In the present invention, the recombinant irisin protein increases the expression of an uncoupling protein (UCP1) in cells, and thus is effective for treating a metabolic disease such as obesity, diabetes, and the like by converting white adipocytes into brown adipocytes, which is known as a function of irisin.

In the present invention, the recombinant irisin protein increases the expression of adiponectin in cells, and thus is effective for treating a metabolic disease, for example, improving insulin resistance, and the like.

In the present invention, the term “brown adipocyte (BA)” refers to a cell that oxidatively decomposes a fatty acid to release its energy as heat, unlike the white adipocytes. This is because a mitochondrial inner membrane protein (uncoupling protein 1; UCP1) specifically expressed by the brown adipocytes uncouples oxidative phosphorylation. In rodents such as mice, BA cells are present around an interscapular region, the posterior neck, the mediastinum, the kidneys, and the like. Also, it is known from the interpretation of UCP1 knockout mice that the BA cells suppress obesity and abnormal glucose tolerance.

The irisin in the composition according to the present invention may be used as it is, or may be used in the form of a salt, preferably a pharmaceutically acceptable salt. In the present invention, the term “pharmaceutically acceptable salt” includes salts derived from pharmaceutically acceptable inorganic or organic acids, or bases.

Examples of suitable acids may include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, gluconic acid, naphthalene-2-sulfonic acid, benzenesulfonic acid, and the like. An acid addition salt may be prepared using a conventional method, for example, by dissolving a compound in an excessive amount of an acidic aqueous solution and precipitating the resulting salt with a water-miscible organic solvent such as methanol, ethanol, acetone, or acetonitrile. Also, the acid addition salt may be prepared by heating the same molar amounts of the compound and an acid or alcohol in water, and then drying the mixture by evaporation or absorbing and filtering the precipitated salt.

The salt derived from a suitable base may include alkali metals such as sodium, potassium, and the like, alkaline earth metals such as magnesium and the like, ammonium, and the like, but the present invention is not limited thereto. The alkali metal or alkaline earth metal salt may be, for example, obtained by dissolving a compound in an excessive amount of a solution of alkali metal hydroxide or alkaline earth metal hydroxide, filtering an insoluble compound salt, and evaporating and drying the filtrate. In this case, it is pharmaceutically suitable to prepare a sodium, potassium, or calcium salt particularly as the metal salt. Also, a silver salt corresponding to the metal salt may be obtained by allowing an alkali metal or alkaline earth metal salt to react with a suitable silver salt (e.g., silver nitrate).

A content of the irisin in the composition of the present invention may be properly adjusted according to the symptoms of a disease, a degree of progression of the symptoms, the condition of a patient, and the like. For example, the content of the irisin may be in a range of 0.0001 to 99.9% by weight, or 0.001 to 50% by weight, based on the total weight of the composition, but the present invention is not limited thereto. The content ratio is a value based on the amount of the dried product from which the solvent is removed.

A pharmaceutically effective amount of the irisin according to the present invention is in a range of 0.001 to 300 mg/day/kg of body weight, preferably 0.01 to 200 mg/day/kg of body weight. However, the pharmaceutically effective amount of the irisin may appropriately vary according to various factors such as a disease and the severity of the disease, the age, weight, health condition, and gender of a patient, a route of administration, a treatment period, and the like.

The pharmaceutical composition according to the present invention may further include a suitable carrier, excipient, and diluent commonly used for preparing the pharmaceutical composition. For example, the excipient may include one or more selected from the group consisting of a diluent, a binder, a disintegrant, a lubricant, an adsorbent, a moisturizing agent, a film-coating material, and a controlled-release additive.

The pharmaceutical composition according to the present invention may be formulated into the form of a preparation for external use, such as a powder, granules, sustained-release granules, enteric granules, a liquid, an eye drop, an elixir, an emulsion, a suspension, a spirit, a troche, aromatic water, a limonade, a tablet, a sustained-release tablet, an enteric tablet, a sublingual tablet, a hard capsule, a soft capsule, a sustained-release capsule, an enteric capsule, a pill, a tincture, a soft extract, a dry extract, a fluid extract, an injection, a capsule, a perfusate, a plaster, a lotion, a paste, a spray, an inhalant, a patch, a sterile injectable solution, an aerosol, or the like, and used according to each of the conventional methods. The preparation for external use may have a formulation such as a cream, a gel, a patch, a spray, an ointment, a plaster, a lotion, a liniment, a paste, a cataplasma, or the like.

The carrier, the excipient, and the diluent, which may be included in the pharmaceutical composition according to the present invention, may include lactose, dextrose, sucrose, an oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

When formulated, the composition may be prepared using a commonly used diluent or excipient such as a filler, an extender, a binder, a wetting agent, a disintegrant, a surfactant, and the like.

Excipients such as corn starch, potato starch, wheat starch, milk sugar, white sugar, glucose, fructose, D-mannitol, precipitated calcium carbonate, synthetic aluminum silicate, calcium hydrogen phosphate, calcium sulfate, sodium chloride, sodium hydrogen carbonate, purified lanolin, microcrystalline cellulose, dextrin, sodium alginate, methyl cellulose, carboxymethyl cellulose sodium, kaolin, urea, colloidal silica gel, hydroxypropyl starch, hydroxypropyl methyl cellulose 1928, 2208, 2906, 2910, propylene glycol, casein, calcium lactate, Primojel, and the like; binders such as gelatin, gum arabic, ethanol, agar powder, cellulose acetate phthalate, carboxymethyl cellulose, carboxymethyl cellulose calcium, glucose, purified water, casein sodium, glycerin, stearic acid, carboxymethyl cellulose sodium, methyl cellulose sodium, methyl cellulose, microcrystalline cellulose, dextrin, hydroxycellulose, hydroxypropyl starch, hydroxymethyl cellulose, purified shellac, gelatinized starch, hydroxypropyl cellulose, hydroxypropyl methyl cellulose, polyvinyl alcohol, polyvinyl pyrrolidone, and the like may be used as the additives of the tablet, the powder, the granule, the capsule, the pill, and the troche according to the present invention. Also, disintegrants such as hydroxypropyl methyl cellulose, corn starch, agar powder, methyl cellulose, bentonite, hydroxypropyl starch, carboxymethyl cellulose sodium, sodium alginate, carboxymethyl cellulose calcium, calcium citrate, sodium lauryl sulfate, a silicic acid anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinyl pyrrolidone, calcium phosphate, gelatinized starch, gum arabic, amylopectin, pectin, sodium polyphosphate, ethyl cellulose, white sugar, magnesium aluminum silicate, a D-sorbitol solution, a hard silicic acid anhydride, and the like; lubricants such as calcium stearate, magnesium stearate, stearic acid, a hydrogenated vegetable oil, talc, lycopodium powder, kaolin, Vaseline, sodium stearate, cacao butter, sodium salicylate, magnesium salicylate, polyethylene glycol 4000, 6000, liquid paraffin, a hydrogenated soybean oil (Lubri wax), aluminum stearate, zinc stearate, sodium lauryl sulfate, magnesium oxide, Macrogol, synthetic aluminum silicate, silicic acid anhydride, a higher fatty acid, a higher alcohol, silicone oil, paraffin oil, a polyethylene glycol fatty acid ester, starch, sodium chloride, sodium acetate, sodium oleate, D/L-leucine, a hard silicic acid anhydride, and the like, may be used.

Water, dilute hydrochloric acid, dilute sulfuric acid, sodium citrate, sucrose monostearates, polyoxyethylene sorbitol fatty acid esters (Tween ester), polyoxyethylene monoalkyl ethers, lanolin ethers, lanolin esters, acetic acid, hydrochloric acid, ammonium hydroxide, ammonium carbonate, potassium hydroxide, sodium hydroxide, prolamine, polyvinyl pyrrolidone, ethyl cellulose, carboxymethyl cellulose sodium, and the like may be used as the additives for liquid formulations according to the present invention.

A solution of white sugar, other sugars, a sweetening agent, and the like may be used as the syrup according to the present invention. A fragrance, a coloring agent, a preservative, a stabilizing agent, a suspending agent, an emulsifier, a thickening agent, and the like may be used, when necessary.

Purified water was used in the emulsion according to the present invention. An emulsifier, a preservative, a stabilizing agent, a fragrance, and the like may be used, when necessary.

Suspending agents such as acacia, tragacanth, methyl cellulose, carboxymethyl cellulose, carboxymethyl cellulose sodium, microcrystalline cellulose, sodium alginate, hydroxypropyl methyl cellulose 1828, 2906, 2910, and the like may be used in the suspension according to the present invention. A surfactant, a preservative, a stabilizing agent, a coloring agent, and a fragrance may be used, when necessary.

The injection according to the present invention may include solvents such as distilled water for injection, 0.9% sodium chloride injection, Ringer's injection, dextrose injection, dextrose+sodium chloride injection, PEG, lactated Ringer injection, ethanol, propylene glycol, non-volatile oil-sesame oil, cottonseed oil, peanut oil, soybean oil, corn oil, ethyl oleate, isopropyl myristate, benzyl benzoate, and the like; dissolution aids such as sodium benzoate, sodium salicylate, sodium acetate, urea, urethane, monoethylacetamide, butazolidine, propylene glycol, Tweens, nicotinamide, hexamine, dimethylacetamide, and the like; isotonic agents such as a weak acid and salts thereof (acetic acid and sodium acetate), a weak base and salts thereof (ammonia and ammonium acetate), an organic compound, a protein, albumin, peptone, gums, and the like; buffers such as sodium chloride and the like; stabilizing agents such as sodium bisulfite (NaHSO₃) carbon dioxide gas, sodium metabisulfite (Na₂S₂O₃), sodium sulfite (Na₂SO₃), nitrogen gas (N₂), ethylene diamine tetraacetic acid, and the like; antioxidants such as sodium bisulfide (0.1%), sodium formaldehyde sulfoxylate, thiourea, ethylene diamine disodium tetraacetate, acetone sodium bisulfite, and the like; analgesics such as benzyl alcohol, chlorobutanol, procaine hydrochloride, glucose, calcium gluconate, and the like; suspending agents such as CMC sodium, sodium alginate, Tween 80, aluminum monostearate, and the like.

Bases such as cacao butter, lanolin, Witepsol, polyethylene glycol, glycerogelatin, methyl cellulose, carboxymethyl cellulose, a mixture of stearic acid and oleic acid, Subanal, cottonseed oil, peanut oil, palm oil, cacao butter+cholesterol, lecithin, lanette wax, glycerol monostearate, Tween or Span, Imhausen, monolene (propylene glycol monostearate), glycerin, Adeps solidus, Buytyrum Tego-G, Cebes Pharma 16, Hexaride Base 95, Cotomar, Hydrokote SP, S-70-XXA, S-70-XX75 (S-70-XX95), Hydrokote 25, Hydrokote 711, Idropostal, Massa estrarium A, AS, B, C, D, E, I, T, Massa-MF, Masuppol, Masuppol-15, Neosuppostal-N, Paramound-B, Supposiro (OSI, OSIX, A, B, C, D, H, L), a suppository base type IV (AB, B, A, BC, BBG, E, BGF, C, D, 299), Suppostal (N, Es), Wecoby (W, R, S, M, Fs), a Tegester triglyceride base (TG-95, MA, 57), and the like may be used in a suppository according to the present invention.

A solid preparation for oral administration includes a tablet, a pill, a powder, granules, a capsule, and the like. Such a solid preparation is prepared by mixing at least one excipient, for example, starch, calcium carbonate, sucrose or lactose, gelatin, and the like with an extract. Also, lubricants such as magnesium stearate, talc, and the like are also used in addition to the simple excipients.

A liquid preparation for oral administration includes a suspension, an oral liquid, an emulsion, syrup, and the like. In addition to the commonly used simple diluents such as water, liquid paraffin, and the like, the liquid preparation may include various excipients, for example, a wetting agent, a sweetening agent, a fragrance, a preservative, and the like.

A preparation for parenteral administration includes a sterile aqueous solution, a non-aqueous solution, a suspension, an emulsion, a freeze-dried preparation, a suppository, and the like. Propylene glycol, polyethylene glycol, a vegetable oil (such as olive oil), an injectable ester (such as ethyl oleate), and the like may be used as a non-aqueous solvent and a suspending agent.

The pharmaceutical composition according to the present invention is administered at a pharmaceutically effective amount. In the present invention, the term “pharmaceutically effective amount” refers to an amount sufficient to treat a disease at a reasonable benefit/risk ratio applicable to medical treatment, and a level of effective dose may be determined according to the type and severity of a patient's disease, the activity of a drug, the sensitivity to the drug, an administration duration, a route of administration and a secretion rate, a treatment period, factors including a concurrently used drug, and other factors well known in the medical field.

The pharmaceutical composition according to the present invention may be administered as an individual therapeutic agent or administered in combination with other therapeutic agents. In this case, the pharmaceutical composition may be administered sequentially or simultaneously with conventional therapeutic agents, and may be administered in single or multiple doses. It is important to administer an amount of the pharmaceutical composition that may realize the maximum effect at the minimum amount without any side effects in consideration of as all the factors described above. In this case, the amount of the pharmaceutical composition may be easily determined by those skilled in the art to which the present invention belongs.

The pharmaceutical composition of the present invention may be administered to a subject via various routes of administration. All modes of administration may be expected, and the pharmaceutical composition may be, for example, administered by oral intake, subcutaneous injection, intraperitoneal administration, intravenous injection, intramuscular injection, paravertebral (intradural) injection, sublingual administration, buccal administration, transrectal administration, cervical insertion, intraocular administration, intraauricular administration, intranasal administration, inhalation, spraying through the mouth or nose, transdermal administration, dermal administration, and the like.

In the pharmaceutical composition of the present invention, the mode of administration may be determined according to the type of drug as an active ingredient in combination with various related factors such as a disease to be treated, a route of administration, the age, gender, and weight of a patient, the severity of a disease, and the like.

In this specification, the term “subject (individual)” refers to a target to which the recombinant irisin protein of the present invention may be administered. More specifically, the subject may include mammals such as a human or a non-human primate, a mouse, a rat, a dog, a cat, a horse, cattle, and the like, but the present invention is not limited thereto.

In the present invention, the term “administration” refers to a process of providing a predetermined amount of the composition of the present invention to a subject using any suitable method.

For the purpose of the present invention, the term “prevention” refers to all types of actions for suppressing or delaying the onset of a metabolic disease by administering the composition of the present invention, and the term “treatment” refers to all types of actions for suppressing the onset or recurrence of a metabolic disease, relieving the symptoms of the metabolic disease, reducing the direct or indirect pathological outcomes of a disorder, slowing the progression rate of the disorder, ameliorating, improving or alleviating the disorder conditions, or refers to an improvement in prognosis. In the present invention, the term “improvement” refers to all types of actions for reducing at least the degree of parameters, for example, symptoms associated with the condition to be treated by administering the composition of the present invention.

A food composition of the present invention includes all types of functional foods, nutritional supplements, health foods, health functional foods and food additives, and the like. The type of the food composition may be prepared in various forms according to conventional methods known in the related art.

For example, the irisin of the present invention may be prepared in the form of a tea, a juice and a drink, and drunk as the health food, or granulated, encapsulated and powdered, and then ingested. Also, the irisin of the present invention may be mixed with a material or an active ingredient known to have an effect on metabolic diseases, and prepared in the form of a composition. For example, the food composition of the present invention may further include a trace mineral, a vitamin, a lipid, a saccharide, and other components known to have a therapeutic effect on the metabolic diseases in addition to the irisin component. The mineral may include a nutrient component required for growth, such as calcium, iron, and the like, and the vitamin may include vitamin C, vitamin E, vitamin B1, vitamin B2, vitamin B6, and the like. The lipid may include alkoxy glycerol, lecithin, or the like, and the saccharide may include a fructooligosaccharide, and the like.

Also, the functional food may be prepared by adding the irisin of the present invention into beverages (including alcoholic beverages), fruits and processed foods thereof (e.g., canned fruits, bottled fruits, jam, marmalade, and the like), fish, meats and processed foods thereof (e.g., ham, sausage, corned beef, and the like), breads and noodles (e.g., Udon, buckwheat noodles, Ramen, spaghetti, macaroni, and the like), fruit juices, various drinks, cookies, taffy, dairy products (e.g., butter, cheese, and the like), edible vegetable fats and oils, margarine, vegetable proteins, retort foods, frozen foods, various condiments (e.g., fermented soybean paste, soy sauce, sauce, and the like), and the like.

In addition, the irisin of the present invention may be prepared in the form of a powder or concentrate, and then used in the form of food additives.

The irisin in the food composition of the present invention may be preferably included at a content of 0.01 to 100% by weight, preferably 0.1 to 50% by weight, based on the total weight of the food, with respect to the total weight of the food composition.

The terms used in the present invention have been selected as widely used general terms as possible in consideration of the functions in the present invention, but this may vary according to the intention or precedent of the person skilled in the art, the emergence of new technologies and the like. In addition, in certain cases, there is also a term arbitrarily selected by the applicant, in which case the meaning will be described in detail in the description of the invention. Therefore, the terms used in the present invention should be defined based on the meanings of the terms and the contents throughout the present invention, rather than the names of the simple terms.

Throughout the specification of the present invention, when any certain part is said to “include” any component, this means that it may further include other components, rather than excluding other components unless otherwise stated. In addition, the terms “approximately,” “substantially,” the like used throughout the specification of the present invention are used at, or in close proximity to, numerical values when manufacturing and material tolerances inherent in the indicated meanings are intended to aid in understanding the present invention. Accurate or absolute figures are used to assist in the prevention of unfair use by unscrupulous infringers.

### MODE FOR INVENTION

Hereinafter, preferred embodiments of the present invention are presented in order to aid in understanding the present invention. However, it should be understood that the following embodiments are given by way of illustration only to more easily understand the present invention, and are not intended to limit the present invention.

### EXAMPLES

### Example 1: Preparation of Recombinant Irisin Vector for Expression in Plant

As shown in FIG. 1, a recombinant plant expression vector for expressing irisin in a plant was constructed. More specifically, genetic information on the human irisin hormone was obtained, a gene was synthesized using a sequence whose expression was optimized in Nicotiana benthamiana (SEQ ID NO: 1). A chaperone binding protein (BiP) (a polynucleotide encoding a signal peptide) (SEQ ID NO: 2), a polynucleotide encoding six continuous histidines (SEQ ID NO: 3), and a polynucleotide encoding irisin were sequentially connected between a CaMV 35S promoter gene and a NOS terminator of a pCAMBIA1300 vector to construct an irisin plant expression vector.

### Example 2: Confirmation of Expression of Recombinant Irisin Protein

2.1: Transient Expression of Plant Expression Vector

The plant expression vector prepared in Example 1 was transformed into an Agrobacterium strain LBA4404 using electroporation. 5 mL of the transformed Agrobacterium was shake-cultured at 28° C. for 16 hours in 5 mL of a yeast extract peptone (YEP) liquid medium (10 g of a yeast extract, 10 g of peptone, 5 g of NaCl, 50 mg/L of kanamycin, and 25 mg/L of rifampicin), and 1 mL of the primary culture broth was seeded in 50 mL of a fresh YEP medium, and then shake-cultured at 28° C. for 6 hours. The Agrobacterium thus cultured was centrifuged (at 7,000 rpm and 4° C. for 5 minutes) to collect the cells, and the cells were resuspended at an optical density (O.D.) of 1.0 in an infiltration buffer (10 mM MES (pH 5.7), 10 mM MgCl₂, 200 μM acetosyringone) at a wavelength of 600 nm. The Agrobacterium suspension was subjected to Agro-infiltration using a method of injecting the suspension into the back of a Nicotiana benthamiana leaf using a syringe from which an injection needle was removed.

2.2: Confirmation of Expression of Recombinant Irisin in Plant

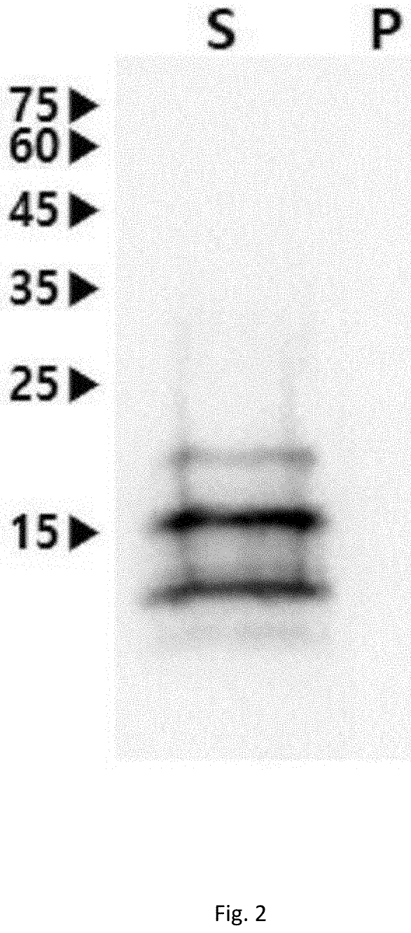

The protein was extracted from the plant leaf prepared in Example 2.1, and centrifuged. Thereafter, the protein in a water-soluble fraction (S) included in the solution, and the protein in a pellet (P) fraction were confirmed by Western blotting. More specifically, 30 μL of each of the fractions was mixed with an SDS sample buffer, and then heated. Then, the protein was separated by size by electrophoresis on a 10% SDS-PAGE gel, and the separated protein was transferred to a PVDF membrane, and then blocked with 5% skim milk. Subsequently, an antibody reacting with six His residues was bound to the membrane, and treated with an ECL solution according to the method provided by the manufacturer to confirm expression of the recombinant irisin. The results are shown in FIG. 2.

As shown in FIG. 2, it was confirmed that the recombinant irisin expressed in the plant was present in the water-soluble fraction, and three types of irisin having different sizes were prepared at the same time.

Based on the results, it can be seen that the irisin expression vector of the present invention may effectively express the recombinant irisin protein in the plant, the irisin prepared using the vector is easily separated and purified because the irisin has high solubility, and the physiological or pharmacological activity of the recombinant protein is effectively maintained because the aggregation of the recombinant protein is suppressed.

### Example 3: Separation and Purification of Recombinant Irisin

200 mL of a protein extraction solution (50 mM sodium phosphate (pH 8.0), 300 mM NaCl, 20 mM imidazole, 0.1% Triton X-100, and a 1× protease inhibitor) was added to 40 g of the Nicotiana benthamiana prepared in Example 2.1, and the tissue was homogenized using a blender. Thereafter, the homogenized tissue was centrifuged at 13,000 rpm and 4° C. for 20 minutes to collect a protein extract.

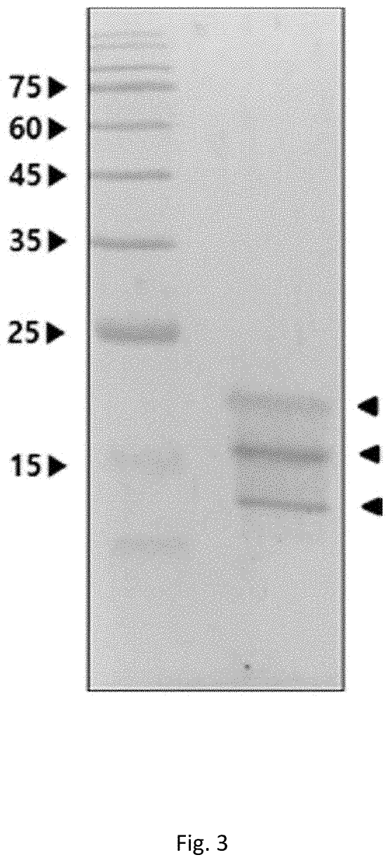

To separate and purify the expressed irisin, the expressed irisin was subjected to affinity chromatography using a column filled with a Ni-NTA agarose resin. A column was filled with 5 mL of the resin, and then equilibrated with 50 mL of a washing solution (50 mM sodium phosphate (pH 8.0), 300 mM NaCl, 20 mM imidazole). The collected protein extract was loaded onto the column, and the resin was then washed with 100 mL of a washing solution. Then, the recombinant protein was eluted with an elution solution (50 mM sodium phosphate (pH 8.0), 300 mM NaCl, 300 mM imidazole). The elution solution including the recombinant protein was replaced with a physiological saline (PBS) solution through a filter having a size of 10 kD, and concentrated to obtain the separated and purified recombinant irisin. The separated and purified protein was subjected to electrophoresis using SDS-PAGE, and then confirmed through Coomassie staining (FIG. 3).

As shown in FIG. 3, it was confirmed that the three recombinant irisin proteins having a size of approximately 13 kD and or so were purified.

The recombinant protein of the present invention was easily purified without any great difference, compared to the existing proteins. Based on these results, it was confirmed that no problem regarding that, when the protein was expressed in the plant, no problem of degraded production efficiency resulting from modification of a sugar structure was found. As a result, it was confirmed that the protein according to the present invention was well produced in the plant.

### Example 4: Confirmation of Glycosylation of Recombinant Irisin by Treatment with Endoglycosidase H

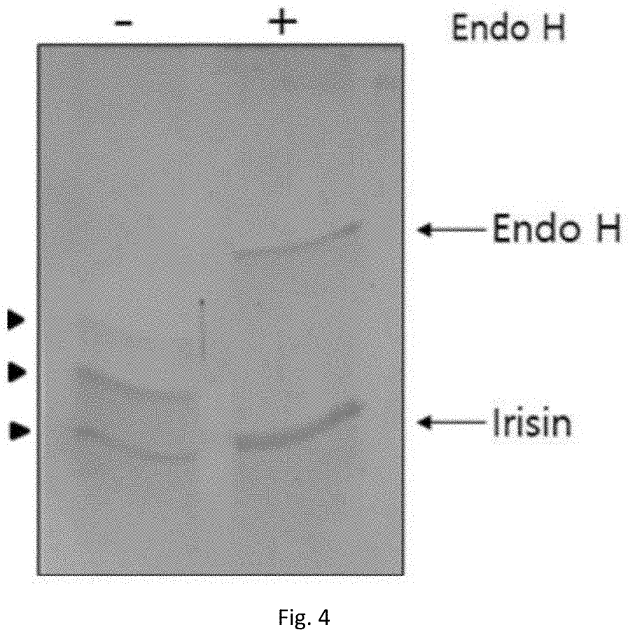

To check whether the separated and purified irisin was glycosylated in the plant, a de-N-glycosylation assay using endoglycosidase H was performed. More specifically, a 10× denaturing buffer (5 SDS, 0.4 M DTT) was added to 1 μg of the recombinant irisin prepared in Example 3, and then heated at 100° C. for 10 minutes. A sodium citrate (pH 5.5) buffer was added thereto so that a final concentration of the buffer was 50 mM, and 50 U of endoglycosidase H was added thereto, and reacted at 37° C. for an hour. For a reaction of the control which did not include endoglycosidase H, an equivalent volume of water was added instead of the endoglycosidase H. When the reaction was completed, the protein was electrophoresed using SDS-PAGE, and a change in molecular weight of the recombinant irisin according to de-N-glycosylation was observed through Coomassie staining (FIG. 4).

As shown in FIG. 4, it can be seen that the irisin with one size was observed as glycans of the irisin was cleaved when the irisin was treated with Endo-H, indicating that the expressed irisin was glycosylated in the plant.

### Example 5: Confirmation of Physiological Activity of Plant-Derived Irisin Protein

3T3-L1 preadipocytes were cultured at 37° C. in a cell culture broth, which was obtained by supplementing a Dulbecco's Modified Eagle medium (DMEM) with 10% FBS, 100 units/mL penicillin, and 100 μg/mL streptomycin, using a humidified CO₂ incubator (5% CO₂/95% air). The adipocytes were differentiated two days after the adipocytes reached a confluence of 100% (post-confluence, day 0), by replacing the culture broth with DMEM supplemented with 10% FBS, 5 μg/mL insulin, 0.25 mM dexamethasone, 0.5 mM 1-methyl-3-isobutylxanthine. After two days, the culture broth was replaced with DMEM supplemented with 10% FBS, 5 μg/mL insulin. After two days, the culture broth was again replaced with DMEM supplemented with 10% FBS. As a result, the adipocytes were differentiated for a total of 10 days, and then used in this experiment. To test an effect on adipose differentiation, the adipocytes were treated with the plant-derived irisin prepared in Example 3 at intervals of two days for 4 days from the day when the differentiation induction was started.

The differentiated adipocytes were treated with the recombinant irisin protein prepared in Example 3 at a concentration of 10 mg/mL. After 48 hours, the expressed protein was extracted from the fat cells, and then subjected to Western blotting.

For analysis of the protein in the 3T3-L1 cells, the cells were treated with a RIPA lysis buffer, and then centrifuged at 13,000 rpm for 15 minutes to separate total proteins in a supernatant. The separated proteins were mixed in a Laemmli sample buffer, and then separated by electrophoresis using SDS-PAGE. Thereafter, the proteins were transferred to a polyvinylidene difluoride (PVDF) membrane. The transferred PVDF membrane was treated with 5% skim milk to block non-specific proteins, cultured overnight at 4° C. with a primary antibody, and then cultured with a secondary antibody at room temperature for an hour. The cultured cells were finally treated with an ECL solution, and an expression level of the protein in LAS-4000 was analyzed. The antibodies used herein were purchased from Cell Signaling Technology (Danvers, Mass., USA) and Santa Cruz Biotechnology (Santa Cruz, Calif., UA).

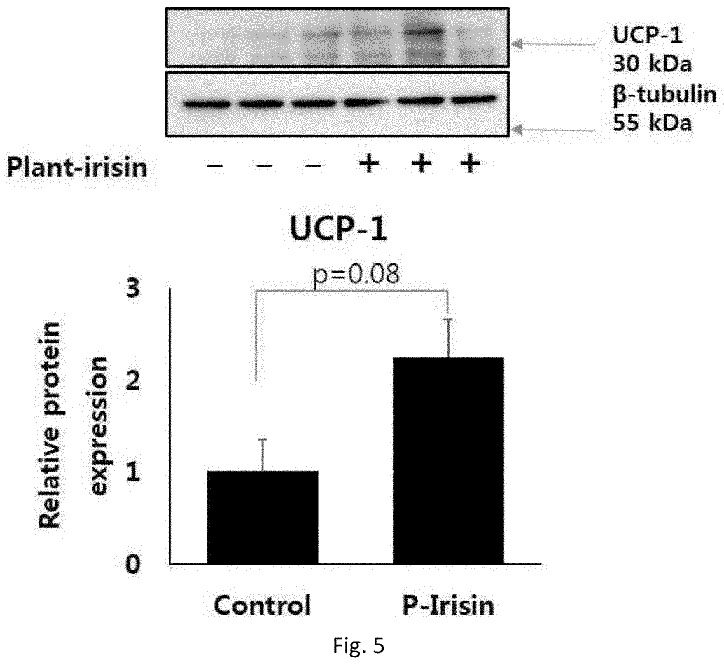

As a result, as shown in FIG. 5, an increase in intracellular expression of the uncoupling protein 1 (UCP1) after treatment with the irisin was observed. UCP 1 is a biomarker that is based on the conversion of white fats into brown fats. Thus, these results suggest that the plant-derived irisin maintained adipocyte browning activity as it is.

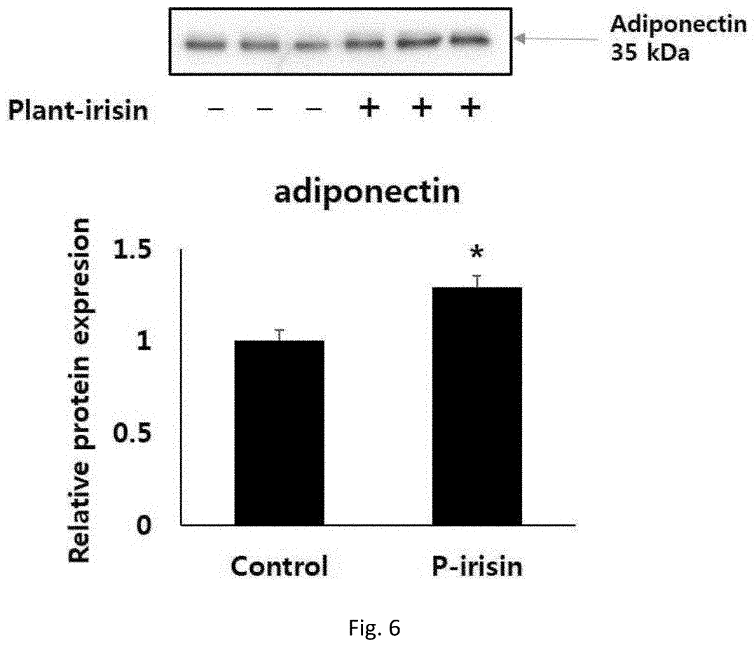

Also, as shown in FIG. 6, an increase in adiponectin secreted from the cells was observed after the cells were treated with the irisin. Adiponectin is one of the representative adipokines secreted from adipocytes, and is known to have anti-diabetic and anti-obesity effects by improving systemic metabolism, for example, by improving insulin resistance. Therefore, this means that the irisin derived from the plant has a function of regulating systemic metabolism by controlling adipokines.

Based on the results, it can be seen that the recombinant irisin protein of the present invention may be effectively expressed in the plant, and may be easily separated and purified because the recombinant irisin protein has high solubility. Also, it can be seen that the recombinant irisin protein of the present invention may be effectively used to treat metabolic diseases because the recombinant irisin protein has an effect of inducing an increase in expression of UCP1 and adiponectin

Hereinafter, the pharmaceutical composition and the food composition according to the present invention will be described with reference to Preparation Examples thereof. However, it should be understood that the following Preparation Examples are given for the purpose of illustration only and are not intended to limit the scope of the present invention.

### Preparation Example 1: Preparation of Pharmaceutical Composition

1.1: Preparation of Powder

| | |
|---|---|
| Recombinant irisin protein | 20 mg |
| Milk sugar | 100 mg |
| Talc | 10 mg |

The components are mixed, and filled in an airtight bag to prepare a powder.

1.2: Preparation of Tablet

| | |
|---|---|
| Recombinant irisin protein | 20 mg |
| Corn starch | 100 mg |
| Milk sugar | 100 mg |
| Stearic acid magnesium | 2 mg |

The components are mixed, and the resulting mixture is tableted to prepare a tablet according to a conventional method for preparing a tablet.

1.3: Preparation of Capsule

| | |
|---|---|
| Recombinant irisin protein | 20 mg |
| Crystalline cellulose | 3 mg |
| Lactose | 14.8 mg |
| Magnesium stearate | 0.2 mg |

The components are mixed, and filled in a gelatin capsule to prepare a capsule according to a conventional method for preparing a capsule.

1.4: Preparation of Injection

| | |
|---|---|
| Recombinant irisin protein | 20 mg |
| Mannitol | 180 mg |
| Sterile distilled water for injection | 2,974 mg |
| Na₂HPo₄2H₂O | 26 mg |

An injection is prepared by adding contents of the components per ampoule (2 mL) according to a conventional method for preparing an injection.

1.5: Preparation of Liquid

| | |
|---|---|
| Recombinant irisin protein | 20 mg |
| Isomerized glucose syrup | 10 g |
| Mannitol | 5 g |
| Purified water | Balance |

A liquid is prepared according to a conventional method for preparing a liquid by adding and dissolving the respective components in purified water, adding a proper amount of a lemon flavor, mixing the above components, adding purified water to adjust the total amount of the resulting mixture to 100 mL, filling the mixture in a brown vial, and sterilizing the mixture.

### Preparation Example 2: Preparation of Health Food

| Recombinant irisin protein | 100 | mg |
|---|---|---|
| Vitamin mixture | proper amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Mineral mixture | proper amount | |
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium citrate | 90 | mg |
| Calcium carbonate | 100 | mg |
| Magnesium chloride | 24.8 | mg |

The composition ratios of the vitamins and the mineral mixture are determined by mixing the components relatively more suitable for the health food, but the blending ratios of the vitamins and the mineral mixture may be arbitrarily modified. The components are mixed, and the resulting mixture is prepared into granules according to a conventional method for preparing a health food. Then, the granules may be used to prepare a health food composition according to the conventional method.

### Preparation Example 3: Preparation of Health Beverage

| | | |
|---|---|---|
| Recombinant irisin protein | 100 | mg |
| Vitamin C | 15 | g |
| Vitamin E (powder) | 100 | g |
| Ferrous lactate | 19.75 | g |
| Zinc oxide | 3.5 | g |
| Nicotinamide | 3.5 | g |
| Vitamin A | 0.2 | g |
| Vitamin B1 | 0.25 | g |
| Vitamin B2 | 0.3 | g |

| | |
|---|---|
| Water | balance |

The components are mixed and heated for approximately an hour while stirring at 85° C., and the prepared solution is then filtered and filled in a sterile 2 L container according to a conventional method for preparing a health beverage. Thereafter, the container is hermetically sealed, sterilized, and then refrigerated. Then, the solution in the container is used to prepare the health beverage composition of the present invention. The composition ratios of the components are determined by mixing the components relatively more suitable for favorite beverages, but the blending ratios of the vitamins and the mineral mixture may be arbitrarily modified according to the regional and national preference such as a class in demand thereof, nations in demand thereof, a purpose of use, or the like.

The above description of the present invention has been given by way of illustration only. Therefore, those skilled in the art to which the present invention pertains will appreciate that the present invention can be embodied in other specific forms without changing the technical spirit or essential features of the present invention. Therefore, it should be understood that the embodiments described above are for the purpose of illustration only, and not intended to be limiting in all respects.

### INDUSTRIAL APPLICABILITY

The recombinant irisin protein of the present invention may be effectively expressed in a plant, and may be easily separated and purified because the recombinant irisin protein has high water solubility. Also, the recombinant irisin protein of the present invention is expected to be effectively used to treat metabolic diseases because the recombinant irisin protein has an effect of inducing an increase in expression of UCP1 and adiponectin. Therefore, the recombinant irisin protein of the present invention is industrially applicable.

1. A recombinant irisin gene whose expression is optimized in plants, the recombinant irisin gene comprising a nucleotide sequence represented by SEQ ID NO: 1.

2. A recombinant expression vector comprising the gene of claim 1.

3. The recombinant expression vector of claim 2, which comprises a structure map as described in FIG. 1.

4. The recombinant expression vector of claim 2, further comprising a gene encoding one or more selected from the group consisting of a chaperone binding protein (BiP) gene and a tagging gene.

5. A transformant transformed with the vector of claim 2.

6. The transformant of claim 5, which is a plant or a plant cell.

7. A method for producing a recombinant irisin protein, comprising:
(a) culturing the transformant of claim 5; and
(b) separating and purifying an irisin protein from the transformant or the culture broth.

8. The method of claim 7, wherein the purification of step (b) is performed using a water-soluble fraction.

9. A composition, comprising the recombinant irisin protein produced by the method of claim 7 as an active ingredient.

10. (canceled)

11. The composition of claim 9, wherein the recombinant irisin protein comprises one or more selected from the group consisting of a glycosylated recombinant irisin protein and a non-glycosylated recombinant irisin protein.

12. The composition of claim 9, wherein the recombinant irisin protein comprises an amino acid sequence set forth in SEQ ID NO: 4.

13-16. (canceled)

17. A method for preventing or treating a metabolic disease, comprising:
administering the recombinant irisin protein produced by the method of claim 7 into a subject.

18. (canceled)

19. (canceled)

20. The method of claim 17, wherein the metabolic disease is selected from the group consisting of obesity, diabetes, hypertension, hyperlipidemia, hypertriglyceridemia, hypercholesterolemia, fatty liver, and atherosclerosis.

21. The method of claim 17, wherein the recombinant irisin protein comprises one or more selected from the group consisting of a glycosylated recombinant irisin protein and a non-glycosylated recombinant irisin protein.

22. The method of claim 17, wherein the recombinant irisin protein comprises an amino acid sequence set forth in SEQ ID NO: 4.


SEQUENCE LISTING (8 sequences)

SEQ ID NO: 1 - DNA, 339 bp, Artificial Sequence
  Irisin
gattctccttcggcaccggttaacgttactgtcaggcatctgaaggctaattcagccgttgtaagctgggatgttttggaggatgaagttgtgattggattcgcgatctctcaacagaagaaagatgtgagaatgttaagatttattcaggaggtcaacacaactaccaggtcatgtgctctttgggatcttgaggaggacacagaatatatagtgcacgtgcaagcaatcagtattcagggtcaatctccagcttccgaaccagtactctttaagacgcctcgtgaggcagagaaaatggctagtaagaataaggacgaagttactatgaaagaatag

SEQ ID NO: 2 - DNA, 251 bp, Artificial Sequence
  Chapherone binding protein(BiP)
atggctcgctcgtttggagctaacagtaccgttgtgttggcgatcatcttcttcggtgagtgattttccgatcttcttctccgatttagatctcctctacattgttgcttaatctcagaaccttttttcgttgttcctggatctgaatgtgtttgtttgcaatttcacgatcttaaaaggttagatctcgattggtattgacgattggaatctttacgatttcaggatgtttatttgcgttgtcctctgca

SEQ ID NO: 3 - DNA, 18 bp, Artificial Sequence
  6 His
caccaccatcaccaccat

SEQ ID NO: 4 - protein, 112 aa, Artificial Sequence
  Irisin AA
DSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKE

SEQ ID NO: 5 - protein, 27 aa, Artificial Sequence
  Chapherone binding protein(BiP) AA
MARSFGANSTVVLAIIFFGCLFALSSA

SEQ ID NO: 6 - protein, 6 aa, Artificial Sequence
  6 His AA
HHHHHH

SEQ ID NO: 7 - DNA, 614 bp, Artificial Sequence
  DNA sequence of full vector
atggctcgctcgtttggagctaacagtaccgttgtgttggcgatcatcttcttcggtgagtgattttccgatcttcttctccgatttagatctcctctacattgttgcttaatctcagaaccttttttcgttgttcctggatctgaatgtgtttgtttgcaatttcacgatcttaaaaggttagatctcgattggtattgacgattggaatctttacgatttcaggatgtttatttgcgttgtcctctgcaggatcccaccaccatcaccaccatgattctccttcggcaccggttaacgttactgtcaggcatctgaaggctaattcagccgttgtaagctgggatgttttggaggatgaagttgtgattggattcgcgatctctcaacagaagaaagatgtgagaatgttaagatttattcaggaggtcaacacaactaccaggtcatgtgctctttgggatcttgaggaggacacagaatatatagtgcacgtgcaagcaatcagtattcagggtcaatctccagcttccgaaccagtactctttaagacgcctcgtgaggcagagaaaatggctagtaagaataaggacgaagttactatgaaagaatag

SEQ ID NO: 8 - protein, 120 aa, Artificial Sequence
  Amino acid sequence of full vector
GSHHHHHHDSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKE